(19) United States
(12) Patent Application Publication
(10) Pub. No.: US 2011/0015393 A1
(43) Pub. Date: Jan. 20, 2011

(54) PHTHALAZINONE COMPOUND

(75) Inventor: Neil HAWKINS, Macclesfield (GB)

Correspondence Address: MICHAEL BEST & FRIEDRICH LLP, 100 East Wisconsin Avenue, Suite 3300, Milwaukee, WI, 53202, US

(73) Assignee: ASTRAZENECA AB, Sodertalje (SE)

(21) Appl. No.: 12/836,903

(22) Filed: Jul. 15, 2010

Related U.S. Application Data
(60) Provisional application No. 61/225,825, filed on Jul. 15, 2009.

(51) Int. Cl. C07D 401/10

(52) U.S. Cl. 544/237

(57) ABSTRACT

4-(4-Fluoro-3-(4-methoxypiperidine-1-carbonyl)benzyl)phthalazin-1(2H)-one as crystalline Form C.

### CROSS-REFERENCE TO RELATED APPLICATIONS

This application claims priority benefits to U.S. Provisional Application No. 61/225,825 filed Jul. 15, 2009, which is incorporated herein by reference in its entirety.

The present invention relates to a crystalline form of a phthalazinone compound and the use of that crystalline form.

The mammalian enzyme PARP-1 (a 113-kDa multidomain protein) has been implicated in the signalling of DNA damage through its ability to recognize and rapidly bind to DNA single or double strand breaks (D'Amours, et al., Biochem. J., 342, 249-268 (1999)).

The family of Poly (ADP-ribose) polymerases now includes around 18 proteins, that all display a certain level of homology in their catalytic domain but differ in their cellular functions (Ame et al., Bioessays., 26(8), 882-893 (2004)). Of this family PARP-1 (the founding member) and PARP-2 are so far the sole enzymes whose catalytic activity are stimulated by the occurrence of DNA strand breaks, making them unique in the family.

It is now known that PARP-1 participates in a variety of DNA-related functions including gene amplification, cell division, differentiation, apoptosis, DNA base excision repair as well as effects on telomere length and chromosome stability (d Adda di Fagagna, et al., Nature Gen., 23(1), 76-80 (1999)).

Studies on the mechanism by which PARP-1 modulates DNA repair and other processes has identified its importance in the formation of poly (ADP-ribose) chains within the cellular nucleus (Althaus, F. R. and Richter, C., ADP-Ribosylation of Proteins: Enzymology and Biological Significance, Springer-Verlag, Berlin (1987)). The DNA-bound, activated PARP-1 utilizes NAD⁺ to synthesize poly (ADP-ribose) on a variety of nuclear target proteins, including topoisomerases, histones and PARP itself (Rhun, et al., Biochem. Biophys. Res. Commun., 245, 1-10 (1998))

Poly (ADP-ribosyl)ation has also been associated with malignant transformation. For example, PARP-1 activity is higher in the isolated nuclei of SV40-transformed fibroblasts, while both leukaemic and colon cancer cells show higher enzyme activity than the equivalent normal leukocytes and colon mucosa (Miwa, et al., Arch. Biochem. Biophys., 181, 313-321 (1977); Burzio, et al., Proc. Soc. Exp. Biol. Med., 149, 933-938 (1975); and Hirai, et al., Cancer Res., 43, 3441-3446 (1983)). More recently in malignant prostate tumours compared to benign prostate cells significantly increased levels of active PARP (predominantly PARP-1) have been identified associated with higher levels of genetic instability (McNealy, et al., Anticancer Res., 23, 1473-1478 (2003)).

A number of low-molecular-weight inhibitors of PARP-1 have been used to elucidate the functional role of poly (ADP-ribosyl)ation in DNA repair. In cells treated with alkylating agents, the inhibition of PARP leads to a marked increase in DNA-strand breakage and cell killing (Durkacz, et al., Nature, 283, 593-596 (1980); Berger, N. A., Radiation Research, 101, 4-14 (1985)).

Subsequently, such inhibitors have been shown to enhance the effects of radiation response by suppressing the repair of potentially lethal damage (Ben-Hur, et al., British Journal of Cancer, 49 (Suppl. VI), 34-42 (1984); Schlicker, et al., Int. J. Radiat. Bioi., 75, 91-100 (1999)). PARP inhibitors have been reported to be effective in radio sensitising hypoxic tumour cells (U.S. Pat. No. 5,032,617; U.S. Pat. No. 5,215,738 and U.S. Pat. No. 5,041,653). In certain tumour cell lines, chemical inhibition of PARP-1 (and PARP-2) activity is also associated with marked sensitisation to very low doses of radiation (Chalmers, Clin. Oncol., 16(1), 29-39 (2004))

Furthermore, PARP-1 knockout (PARP −/−) animals exhibit genomic instability in response to alkylating agents and γ-irradiation (Wang, et al., Genes Dev., 9, 509-520 (1995); Menissier de Murcia, et al., Proc. Natl. Acad. Sci. USA, 94, 7303-7307 (1997)). More recent data indicates that PARP-1 and PARP-2 possess both overlapping and non-redundant functions in the maintenance of genomic stability, making them both interesting targets (Menissier de Murcia, et al., EMBO. J., 22(9), 2255-2263 (2003)).

PARP inhibition has also recently been reported to have antiangiogenic effects. Where dose dependent reductions of VEGF and basic-fibroblast growth factor (bFGF)-induced proliferation, migration and tube formation in HUVECS has been reported (Rajesh, et al., Biochem. Biophys. Res. Comm., 350, 1056-1062 (2006)).

A role for PARP-1 has also been demonstrated in certain vascular diseases, septic shock, ischaemic injury and neurotoxicity (Cantoni, et al., Biochim. Biophys. Acta, 1014, 1-7 (1989); Szabo, et al., J. Clin. Invest, 100, 723-735 (1997)). Oxygen radical DNA damage that leads to strand breaks in DNA, which are subsequently recognised by PARP-1, is a major contributing factor to such disease states as shown by PARP-1 inhibitor studies (Cosi, et al., J. Neurosci. Res., 39, 38-46 (1994); Said, et al., Proc. Natl. Acad. Sci. U.S.A., 93, 4688-4692 (1996)). More recently, PARP has been demonstrated to play a role in the pathogenesis of haemorrhagic shock (Liaudet, et al., Proc. Natl. Acad. Sci. U.S.A., 97(3), 10203-10208 (2000)).

It has also been demonstrated that efficient retroviral infection of mammalian cells is blocked by the inhibition of PARP-1 activity. Such inhibition of recombinant retroviral vector infections was shown to occur in various different cell types (Gaken, et al., J. Virology, 70(6), 3992-4000 (1996)). Inhibitors of PARP-1 have thus been developed for the use in anti-viral therapies and in cancer treatment (WO 91/18591).

Moreover, PARP-1 inhibition has been speculated to delay the onset of aging characteristics in human fibroblasts (Rattan and Clark, Biochem. Biophys. Res. Comm., 201(2), 665-672 (1994)). This may be related to the role that PARP plays in controlling telomere function (d'Adda di Fagagna, et al., Nature Gen., 23(1), 76-80 (1999)).

PARP inhibitors are also thought to be relevant to the treatment of inflammatory bowel disease (Szabo C., Role of Poly(ADP-Ribose) Polymerase Activation in the Pathogenesis of Shock and Inflammation, In PARP as a Therapeutic Target; Ed J. Zhang, 2002 by CRC Press; 169-204), ulcerative colitis (Zingarelli, B, et al., Immunology, 113(4), 509-517 (2004)) and Crohn's disease (Jijon, H. B., et al., Am. J. Physiol. Gastrointest. Liver Physiol., 279, G641-G651 (2000).

Co-pending International Application PCT/GB2009/000181 filed 23 Jan. 2009, now published as WO 2009/093032, discloses compounds of the formula:

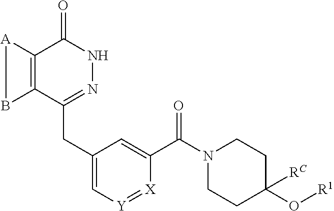

wherein:
A and B together represent an optionally substituted, fused aromatic ring;
X and Y are selected from CH and CH, CF and CH, CH and CF and N and CH respectively;
R^{C} is selected from H, C₁₋₄ alkyl; and
R¹ is selected from C₁₋₇ alkyl, C₃₋₂₀ heterocyclyl and C₅₋₂₀ aryl, which groups are optionally substituted; or
R^{C} and R¹ together with the carbon and oxygen atoms to which they are attached form a spiro-C₅₋₇ oxygen-containing heterocyclic group, which is optionally substituted or fused to a C₅₋₇ aromatic ring.
4-(4-Fluoro-3-(4-methoxypiperidine-1-carbonyl)benzyl)phthalazin-1(2H)-one (compound 1) disclosed in PCT/GB2009/000181:

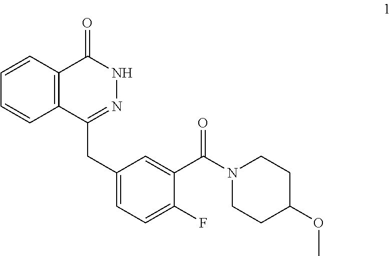

is of particular interest.

Crystalline forms of compound 1 (Forms A and B) are disclosed in PCT/GB2009/000181.

Particular crystalline forms of compound 1 may have advantageous properties, for example with regard to their solubility and/or their stability and/or their bioavailability and/or their impurity profile and/or their filtration characteristics and/or their drying characteristics and/or their lack of hygroscopicity, and/or they may be easier to handle and/or micronise and/or form into tablets.

Accordingly, the first aspect of the present invention provides 4-(4-fluoro-3-(4-methoxypiperidine-1-carbonyl)benzyl)phthalazin-1(2H)-one (compound 1) substantially as crystalline Form C.

“Substantially as crystalline Form C” as used above, means that at least 50% by weight of compound 1 is in Form C, preferably at least 70% by weight, 80% or 90% by weight. In some embodiments, at least 95% by weight, 99% by weight or even 99.5% or more by weight may be in Form C.

Compound 1 Form C is characterised in providing at least one of the following 2θ values measured using CuKa radiation in X-ray powder diffraction: 19.3° and 18.5°. Compound 1 Form C may also characterised in providing an X-ray powder diffraction pattern, substantially as shown in FIG. 1. The ten most prominent peaks are shown in Table A:

TABLE A
Ten most Prominent X-Ray Powder Diffraction
peaks for Compound 1 Form C

| Angle 2- | | Relative |
|---|---|---|
| Theta (2θ) | Intensity % | Intensity |
| 19.277 | 100.0 | vs |
| 18.508 | 99.8 | vs |
| 18.887 | 92.2 | vs |
| 13.060 | 81.4 | vs |
| 21.420 | 72.2 | vs |
| 22.826 | 68.2 | vs |
| 13.377 | 65.9 | vs |
| 10.467 | 62.6 | vs |
| 23.180 | 61.6 | vs |
| 16.505 | 59.6 | vs |

vs = very strong

Therefore, according to the present invention there is provided a crystalline form, Form C, which has an X-ray powder diffraction pattern with at least one specific peak at about 2-theta=19.3°.

According to a further aspect of the present invention there is provided a crystalline form, Form C, which has an X-ray powder diffraction pattern with at least one specific peak at about 2-theta=18.5°.

According to a further aspect of the present invention there is provided a crystalline form, Form C, which has an X-ray powder diffraction pattern with at least two specific peaks at about 2-theta=19.3° and 18.5°.

According to a further aspect of the present invention there is provided a crystalline form, Form C, which has an X-ray powder diffraction pattern with specific peaks at about 2-theta=19.3, 18.5, 18.9, 22.8, 10.5, 23.2°.

According to a further aspect of the present invention there is provided a crystalline form, Form C, which has an X-ray powder diffraction pattern with specific peaks at about 2-theta=19.3, 18.5, 18.9, 13.0, 21.4, 22.8, 13.4, 10.5, 23.2, 16.5°.

According to a further aspect of the present invention there is provided crystalline form, Form C which has an X-ray powder diffraction pattern substantially the same as the X-ray powder diffraction pattern shown in FIG. 1.

According to a further aspect of the present invention there is provided crystalline form, Form C, which has an X-ray powder diffraction pattern with at least one specific peak at 2-theta=19.3° plus or minus 0.5° 2-theta.

According to a further aspect of the present invention there is provided a crystalline form, Form C, which has an X-ray powder diffraction pattern with at least one specific peak at 2-theta=18.5° plus or minus 0.5° 2-theta.

According to a further aspect of the present invention there is provided a crystalline form, Form C, which has an X-ray powder diffraction pattern with at least two specific peaks at 2-theta=19.3° and 18.5° wherein said values may be plus or minus 0.5° 2-theta.

According to a further aspect of the present invention there is provided a crystalline form, Form C, which has an X-ray powder diffraction pattern with specific peaks at 2-theta=19.3, 18.5, 18.9, 22.8, 10.5, 23.2° wherein said values may be plus or minus 0.5° 2-theta.

According to a further aspect of the present invention there is provided a crystalline form, Form C, which has an X-ray powder diffraction pattern with specific peaks at 2-theta=19.3, 18.5, 18.9, 13.0, 21.4, 22.8, 13.4, 10.5, 23.2, 16.5° wherein said values may be plus or minus 0.5° 2-theta.

According to a further aspect of the present invention there is provided a crystalline form, Form C, which has an X-ray powder diffraction pattern with at least one specific peak at 2-theta=19.3°.

According to a further aspect of the present invention there is provided a crystalline form, Form C, which has an X-ray powder diffraction pattern with at least one specific peak at 2-theta=18.5°.

According to a further aspect of the present invention there is provided a crystalline form, Form C, which has an X-ray powder diffraction pattern with at least two specific peaks at 2-theta=19.3° and 18.5°.

According to a further aspect of the present invention there is provided a crystalline form, Form C, which has an X-ray powder diffraction pattern with specific peaks at 2-theta=19.3, 18.5, 18.9, 22.8, 10.5, 23.2°.

According to a further aspect of the present invention there is provided crystalline form, Form C, which has an X-ray powder diffraction pattern with specific peaks at 2-theta=19.3, 18.5, 18.9, 13.0, 21.4, 22.8, 13.4, 10.5, 23.2, 16.5°

According to a further aspect of the present invention there is provided crystalline form, Form C, which has an X-ray powder diffraction pattern as shown in FIG. 1.

Form C of compound 1 may also be characterized using DSC (differential scanning calorimetry). DSC analysis of Compound 1 Form C shows a single event with an onset at 150.7° C. and a peak at 154.9° C. (FIG. 2) when heated at 10° C. per minute.

Thus DSC analysis shows Compound 1 Form C is a high melting solid with an onset of melting at about 150.7° C. and a peak at 154.9° C. when heated at 10° C. per minute.

When it is stated that the present invention relates to a crystalline form of Compound 1 Form C, the degree of crystallinity is conveniently greater than about 60%, more conveniently greater than about 80%, preferably greater than about 90% and more preferably greater than about 95%. Most preferably the degree of crystallinity is greater than about 98%.

Compound 1 Form C provides X-ray powder diffraction patterns substantially the same as the X-ray powder diffraction patterns shown in FIG. 1 and has substantially the ten most prominent peaks (angle 2-theta values) shown in Table A. It will be understood that the 2-theta values of the X-ray powder diffraction pattern may vary slightly from one machine to another or from one sample to another, and so the values quoted are not to be construed as absolute.

It is known that an X-ray powder diffraction pattern may be obtained which has one or more measurement errors depending on measurement conditions (such as equipment or machine used). In particular, it is generally known that intensities in an X-ray powder diffraction pattern may fluctuate depending on measurement conditions. Therefore it should be understood that the Compound 1 Form C of the present invention is not limited to the crystals that provide X-ray powder diffraction patterns identical to the X-ray powder diffraction pattern shown in FIG. 1, and any crystals providing X-ray powder diffraction patterns substantially the same as those shown in FIG. 1 fall within the scope of the present invention. A person skilled in the art of X-ray powder diffraction is able to judge the substantial identity of X-ray powder diffraction patterns.

Persons skilled in the art of X-ray powder diffraction will realise that the relative intensity of peaks can be affected by, for example, grains above 30 microns in size and non-unitary aspect ratios, which may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values. (Jenkins, R & Snyder, R. L. ‘Introduction to X-Ray Powder Diffractometry’ John Wiley & Sons 1996; Bunn, C. W. (1948), Chemical Crystallography, Clarendon Press, London; Klug, H. P. & Alexander, L. E. (1974), X-Ray Diffraction Procedures).

Generally, a measurement error of a diffraction angle in an X-ray powder diffractogram is approximately plus or minus 0.5° 2-theta, and such degree of a measurement error should be taken into account when considering the X-ray powder diffraction pattern in FIG. 1 and when reading Table A. Furthermore, it should be understood that intensities might fluctuate depending on experimental conditions and sample preparation (preferred orientation).

A second aspect of the present invention provides a pharmaceutical composition comprising the compound of the first aspect and a pharmaceutically acceptable carrier or diluent.

A third aspect of the present invention provides the use of the compound of the first aspect in a method of treatment of the human or animal body.

A fourth aspect of the present invention provides the use of the compound of the first aspect of the invention in the preparation of a medicament for:

(a) preventing poly(ADP-ribose) chain formation by inhibiting the activity of cellular PARP (PARP-1 and/or PARP-2);
(b) the treatment of: vascular disease; septic shock; ischaemic injury, both cerebral and cardiovascular; reperfusion injury, both cerebral and cardiovascular; neurotoxicity, including acute and chronic treatments for stroke and Parkinson's disease; haemorraghic shock; inflammatory diseases, such as arthritis, inflammatory bowel disease, ulcerative colitis and Crohn's disease; multiple sclerosis; secondary effects of diabetes; as well as the acute treatment of cytoxicity following cardiovascular surgery or diseases ameliorated by the inhibition of the activity of PARP;
(c) use as an adjunct in cancer therapy or for potentiating tumour cells for treatment with ionizing radiation or chemotherapeutic agents.

In particular, the compound of the first aspect of the invention can be used in anti-cancer combination therapies (or as adjuncts) along with alkylating agents, such as methyl methanesulfonate (MMS), temozolomide and dacarbazine (DTIC), also with topoisomerase-1 inhibitors like Topotecan, Irinotecan, Rubitecan, Exatecan, Lurtotecan, Gimetecan, Diflomotecan (homocamptothecins); as well as 7-substituted non-silatecans; the 7-silyl camptothecins, BNP 1350; and non-camptothecin topoisomerase-I inhibitors such as indolocarbazoles also dual topoisomerase-I and II inhibitors like the benzophenazines, XR 11576/MLN 576 and benzopyridoindoles. Such combinations could be given, for example, as intravenous preparations or by oral administration as dependent on the preferred method of administration for the particular agent.

Other further aspects of the invention provide for the treatment of disease ameliorated by the inhibition of PARP, comprising administering to a subject in need of treatment a therapeutically-effective amount of the compound of the first aspect, preferably in the form of a pharmaceutical composition and the treatment of cancer, comprising administering to a subject in need of treatment a therapeutically-effective amount of a compound as defined in the first aspect in combination, preferably in the form of a pharmaceutical composition, simultaneously or sequentially with radiotherapy (ionizing radiation) or chemotherapeutic agents.

In further aspects of the present invention, the compounds may be used in the preparation of a medicament for the treatment of cancer which is deficient in Homologous Recombination (HR) dependent DNA double strand break (DSB) repair activity, or in the treatment of a patient with a cancer which is deficient in HR dependent DNA DSB repair activity, comprising administering to said patient a therapeutically-effective amount of the compound.

The HR dependent DNA DSB repair pathway repairs double-strand breaks (DSBs) in DNA via homologous mechanisms to reform a continuous DNA helix (K. K. Khanna and S. P. Jackson, Nat. Genet. 27(3): 247-254 (2001)). The components of the HR dependent DNA DSB repair pathway include, but are not limited to, ATM (NM_{—}000051), RAD51 (NM_{—}002875), RAD51L1 (NM_{—}002877), RAD51 C(NM_{—}002876), RAD51L3 (NM_{—}002878), DMC1 (NM_{—}007068), XRCC2 (NM_{—}005431), XRCC3 (NM_{—}005432), RAD52 (NM_{—}002879), RAD54L (NM_{—}003579), RAD54B (NM_{—}012415), BRCA1 (NM_{—}007295), BRCA2 (NM_{—}000059), RAD50 (NM_{—}005732), MRE11A (NM_{—}005590) and NBS1 (NM_{—}002485). Other proteins involved in the HR dependent DNA DSB repair pathway include regulatory factors such as EMSY (Hughes-Davies, et al., Cell, 115, pp 523-535). HR components are also described in Wood, et al., Science, 291, 1284-1289 (2001).

A cancer which is deficient in HR dependent DNA DSB repair may comprise or consist of one or more cancer cells which have a reduced or abrogated ability to repair DNA DSBs through that pathway, relative to normal cells i.e. the activity of the HR dependent DNA DSB repair pathway may be reduced or abolished in the one or more cancer cells.

The activity of one or more components of the HR dependent DNA DSB repair pathway may be abolished in the one or more cancer cells of an individual having a cancer which is deficient in HR dependent DNA DSB repair. Components of the HR dependent DNA DSB repair pathway are well characterised in the art (see for example, Wood, et al., Science, 291, 1284-1289 (2001)) and include the components listed above.

In some preferred embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535) or by an epigenetic mechanism such as gene promoter methylation.

BRCA1 and BRCA2 are known tumour suppressors whose wild-type alleles are frequently lost in tumours of heterozygous carriers (Jasin M., Oncogene, 21(58), 8981-93 (2002); Tutt, et al., Trends Mol. Med., 8(12), 571-6, (2002)). The association of BRCA1 and/or BRCA2 mutations with breast cancer is well-characterised in the art (Radice, P. J., Exp. Clin. Cancer Res., 21(3 Suppl), 9-12 (2002)). Amplification of the EMSY gene, which encodes a BRCA2 binding factor, is also known to be associated with breast and ovarian cancer.

Carriers of mutations in BRCA1 and/or BRCA2 are also at elevated risk of cancer of the ovary, prostate and pancreas.

In some preferred embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Janatova M., et al., Neoplasma, 50(4), 246-50 (2003). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

### FIGURES

FIG. 1 shows a representative powder XRD pattern of compound 1 as Form C;

FIG. 2 shows a representative DSC trace of compound 1 as Form C obtained by heating from 25° C. to 325° C. at 10° C. per minute.

FIG. 3 shows a representative powder XRD pattern of compound 1 as Form A;

FIG. 4 shows a representative DSC trace of compound 1 as Form A obtained by heating from 25° C. to 325° C. at 10° C. per minute.

### USE

The present invention provides compound 1 as Form C as an active compound, specifically, active in inhibiting the activity of PARP.

The term “active” as used herein, pertains to the compound which is capable of inhibiting PARP activity, and specifically includes both compounds with intrinsic activity (drugs) as well as prodrugs of such compounds, which prodrugs may themselves exhibit little or no intrinsic activity.

One assay which may conveniently be used in order to assess the PARP inhibition offered by a particular compound is described in the examples below.

The present invention further provides a method of inhibiting the activity of PARP in a cell, comprising contacting said cell with an effective amount of the active compound, preferably in the form of a pharmaceutically acceptable composition. Such a method may be practiced in vitro or in vivo.

For example, a sample of cells may be grown in vitro and the active compound brought into contact with said cells, and the effect of the compound on those cells observed. As examples of “effect”, the amount of DNA repair effected in a certain time may be determined. Where the active compound is found to exert an influence on the cells, this may be used as a prognostic or diagnostic marker of the efficacy of the compound in methods of treating a patient carrying cells of the same cellular type.

The term “treatment”, as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “adjunct” as used herein relates to the use of the active compound in conjunction with known therapeutic means. Such means include cytotoxic regimes of drugs and/or ionising radiation as used in the treatment of different cancer types. In particular, the active compound is a member of a class known to potentiate the actions of a number of cancer chemotherapy treatments, which include the topoisomerase class of poisons (e.g. topotecan, irinotecan, rubitecan), most of the known alkylating agents (e.g. DTIC, temozolamide) and platinum based drugs (e.g. carboplatin, cisplatin) used in treating cancer.

The active compound may also be used as cell culture additives to inhibit PARP, for example, in order to sensitize cells to known chemotherapeutic agents or ionising radiation treatments in vitro.

The active compound may also be used as part of an in vitro assay, for example, in order to determine whether a candidate host is likely to benefit from treatment with the compound in question.

### Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human.

### Formulations

While it is possible for the active compound to be administered alone, it is preferable to present it as a pharmaceutical composition (e.g., formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing the active compound, as defined above, together with one or more pharmaceutically acceptable carriers, excipients, buffers, adjuvants, stabilisers, or other materials, as described herein, such that the active compound remains as Form C.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, “Handbook of Pharmaceutical Additives”, 2nd Edition (eds. M. Ash and I. Ash), 2001 (Synapse Information Resources, Inc., Endicott, N.Y., USA), “Remington's Pharmaceutical Sciences”, 20th edition, pub. Lippincott, Williams & Wilkins, 2000; and “Handbook of Pharmaceutical Excipients”, 2nd edition, 1994.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of suspensions, tablets, granules, powders, capsules, cachets, pills or pastes.

Formulations suitable for oral administration (e.g., by ingestion) may be presented as discrete units such as capsules, cachets or tablets, each containing a predetermined amount of the active compound; as a powder or granules; as a suspension in an aqueous or non-aqueous liquid; or as a paste.

A tablet may be made by conventional means, e.g. compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

A capsule may include the active compound in suspension.

Formulations suitable for topical administration (e.g. transdermal, intranasal, ocular, buccal, and sublingual) may be formulated as a paste.

Formulations suitable for topical administration to the eye also include eye drops wherein the active compound is suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

### Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

### EXAMPLES

### General Methods

### X-Ray Powder Diffraction

TABLE B

| % Relative Intensity* | Definition | |
|---|---|---|
| 25-100 | vs | (very strong) |
| 10-25 | s | (strong) |
| 3-10 | m | (medium) |
| 1-3 | w | (weak) |

*The relative intensities are derived from diffractograms measured with fixed slits
Analytical Instrument: Bruker D4.

The X-ray powder diffraction spectra were determined by mounting a sample of the crystalline material on a Bruker single silicon crystal (SSC) wafer mount and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5406 angstroms. The collimated X-ray source was passed through an automatic variable divergence slit set at V20 and the reflected radiation directed through a 5.89 mm antiscatter slit and a 9.55 mm detector slit. The sample was exposed for 0.03 seconds per 0.00570° 2-theta increment (continuous scan mode) over the range 2 degrees to 40 degrees 2-theta in theta-theta mode. The running time was 3 minutes and 36 seconds. The instrument was equipped with a Position sensitive detector (Lynxeye). Control and data capture was by means of a Dell Optiplex 686 NT 4.0 Workstation operating with Diffract+software. Persons skilled in the art of X-ray powder diffraction will realise that the relative intensity of peaks can be affected by, for example, grains above 30 microns in size and non-unitary aspect ratios that may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values.

### Differential Scanning Calorimetry

### Analytical Instrument: TA Instruments Q1000 DSC.

Typically less than 5 mg of material contained in a standard aluminium pan fitted with a lid was heated over the temperature range 25° C. to 325° C. at a constant heating rate of 10° C. per minute. A purge gas using nitrogen was used—flow rate 100 ml per minute.

### Analytical LC-MS

LC-MS data was generated on a system where the HPLC component comprised generally either an Agilent 1100, Waters Alliance HT (2790 & 2795) equipment or an HP1100 pump and Diode Array with CTC autosampler and was run on a Phenomenex Gemini C18 5 mm, 50×2 mm column (or similar) eluting with either acidic eluent (for example, using a gradient, over 4 minutes, between 0-95% water/acetonitrile with 5% of a 1% formic acid in 50:50 water:acetonitrile (v/v) mixture; or using an equivalent solvent system with methanol instead of acetonitrile), or basic eluent (for example, using a gradient, over 4 minutes, between 0-95% water/acetonitrile with 5% of a 0.1% 880 Ammonia in acetonitrile mixture); and the MS component comprised generally a Waters ZQ mass spectrometer scanning over an appropriate mass range. Chromatograms for Electrospray (ESI) positive and negative Base Peak Intensity, and UV Total Absorption Chromatogram from 220-300 nm, are generated and values for m/z are given; generally, only ions which indicate the parent mass are reported and unless otherwise stated the value quoted is the (M+H)⁺ for positive ion mode and (M−H)− for negative ion mode

### NMR Spectra

Where given NMR data was determined at 400 MHz using, for example, a Bruker DPX-400 spectrometer and is in the form of delta values, for major diagnostic protons, given in parts per million (ppm). Solvents used were CDCl₃ (with tetramethylsilane (TMS) as an internal standard) or DMSO-d₆ unless otherwise indicated; the following abbreviations have been used: s, singlet; d, doublet; t, triplet; q, quartet; m, multiplet; br, broad.

### Example 1

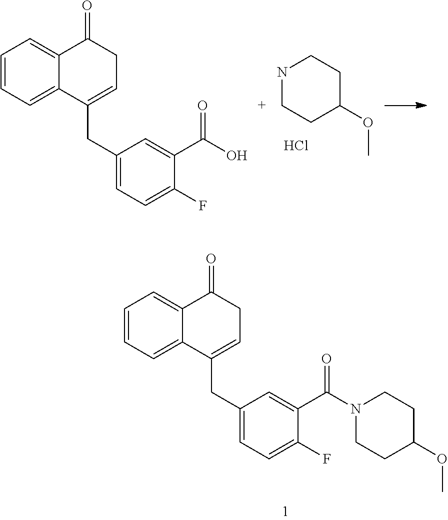

1-(3-Dimethylaminopropyl)-3-ethylcarbodiimide hydrochloride (231 g, 1206.97 mmol) was added to 4-methoxypiperidine hydrochloride (183 g, 1206.97 mmol), 2-fluoro-5-((4-oxo-3,4-dihydrophthalazin-1-yl)methyl)benzoic acid (300 g, 1005.80 mmol) and 4-dimethylaminopyridine (30.7 g, 251.45 mmol) in DCM (4 L) at 23° C. The resulting suspension was stirred at room temperature over night. The reaction mixture was washed with 2M HCl (5 L) and 50% saturated sodium carbonate (3 L) before being dried over MgSO₄, filtered and reduced in-vacuo to give the crude product. This was then slurried in ˜750 ml of ethyl acetate for 5 days, and then filtered and dried at 45° C. for 5 hours to afford 4-(4-fluoro-3-(4-methoxypiperidine-1-carbonyl)benzyl)phthalazin-1(2H)-one (compound 1)(290 g, 72.9%).

¹H NMR (400.132 MHz, DMSO) δ 1.26-1.35 (1H, m), 1.40-1.49 (1H, m), 1.69-1.73 (1H, m), 1.84-1.89 (1H, m), 2.99-3.07 (1H, m), 3.25 (3H, s), 3.27-3.34 (2H, m), 3.39-3.44 (1H, m), 3.86-3.95 (1H, m), 4.33 (2H, s), 7.19-7.24 (1H, m), 7.33-7.35 (1H, m), 7.39-7.43 (1H, m), 7.81-7.91 (2H, m), 7.97 (1H, d), 8.27 (1H, d), 12.57 (1H, s); m/z (ES⁺) (M+H)⁺=396.31; HPLC t^{R}=1.90 min.

FIG. 1 shows the powder XRD pattern of the material produced, which is in Form C.

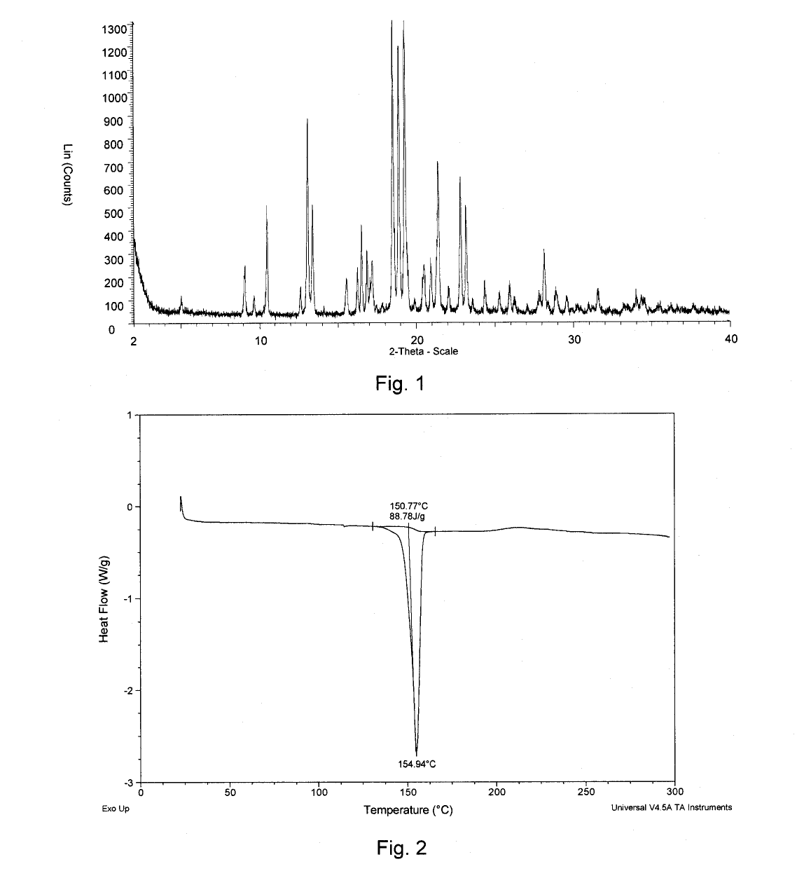

FIG. 2 shows the DSC analysis of the material produced.

### Comparative Example

O-Benzotriazol-1-yl-tetramethyluronium hexafluorophosphate (45.5 g, 119.86 mmol) was added portionwise to a solution of 2-fluoro-5-((4-oxo-3,4-dihydrophthalazin-1-yl)methyl)benzoic acid (1) (27.5 g, 92.20 mmol), 4-methoxypiperidine (11.68 g, 101.42 mmol) and triethylamine (30.8 mL, 221.28 mmol) in DMA (450 mL) at 20° C. under nitrogen. The resulting solution was stirred at 20° C. for 21 hours. The solution was poured into water (2.5 litres) and extracted with EtOAc (×3), the combined extracts washed with brine (×3), dried (MgSO₄), filtered and evaporated to a gum. The crude product was purified by flash silica chromatography, elution gradient 0 to 100% EtOAc in isohexane. Pure fractions were evaporated to dryness and slurried with EtOAc to afford 4-(4-fluoro-3-(4-methoxypiperidine-1-carbonyl)benzyl)phthalazin-1(2H)-one (1)(22.45 g, 61.6%) as a white solid after filtration and vacuum drying.

FIG. 3 shows the powder XRD pattern of the material produced, which is Compound 1 in Form A.

| Angle 2- | | Relative |
|---|---|---|
| Theta (2θ) | Intensity % | Intensity |
| 4.9 | 60 | vs |
| 9.9 | 17 | s |
| 13.2 | 13 | s |
| 14.9 | 15 | s |
| 15.5 | 19 | s |
| 17.4 | 40 | vs |
| 17.8 | 13 | s |
| 19.9 | 100 | vs |
| 24.4 | 12 | s |
| 24.9 | 10 | s |

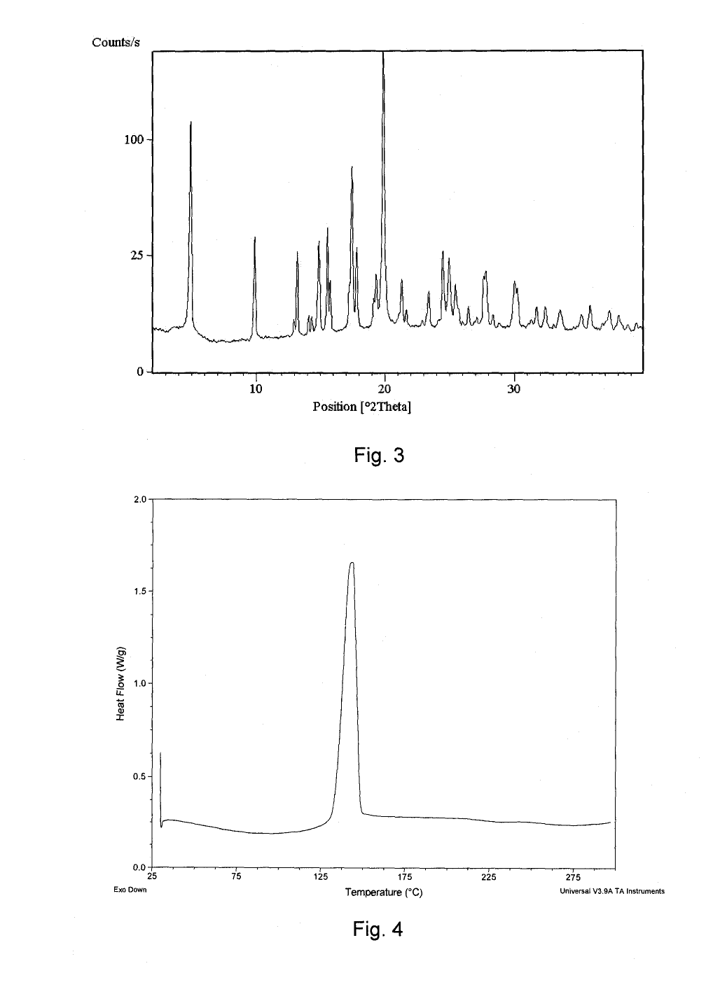

FIG. 4 shows the DSC analysis of the material produced. DSC analysis shows Compound 1, Form A is a high melting solid with an onset of melting at 134° C. and a peak at 143° C. when heated at a rate of 10° C. per minute.

4-(4-fluoro-3-(4-methoxypiperidine-1-carbonyl)benzyl)phthalazin-1(2H)-one (compound 1) can also be synthesized by heating 2-fluoro-5-((4-oxo-3,4-dihydrophthalazin-1-yl)methyl)benzoic acid with 1.5 equivalents of 1,1′-carbonyldiimidazole (CDI) in acetonitrile at 45° C. for about 2 hours, followed by addition of 4-methoxypiperidine hydrochloride (1.2 to 1.4 equivalents) in portions and agitating for several hours at 45° C. The desired compound can then be extracted by first adding butyronitrile, followed by vacuum distillation and a second addition of butyronitrile followed by washing with aqueous base, aqueous acid and water. Form A can be obtained by including a seeding step with existing material in Form A.

### Example 2

### a) Production of a Mixture of Forms

80.8 g of Compound 1, Form A and 289.6 g of Compound 1, Form C were combined and initially suspended in toluene (750 ml). This was removed by rotary evaporation to drive off any water, then the solid was suspended in ethyl acetate (750 ml) and this process was repeated a further 2 time to remove the toluene. The solid was then suspended in ethyl acetate (750 ml) and slurried for 2 hours before the addition of iso-hexane (250 ml). This was then slurried for 48 hours before filtering and washing with iso-hexane (2×250 ml). The product was the dried in a vacuum oven at 50° C. on high vacuum over night to give a single uniform batch of compound 1 (359 g, 97%) as a white crystalline solid.

Analysis by X-ray powder diffraction showed the material to be crystalline and a mixture of forms, and the DSC analysis had endothermic events at 133° C. and 150° C. (onset), showing the material still to be a mixture of forms A and C.

### b) Slurrying to Produce Form C

Approximately 20 mg of the mixed form material was placed in a vial with a magnetic flea, and approximately 2 ml of cyclohexane added. The vial was then sealed tightly with a cap and left to stir on a magnetic stirrer plate. After 7 days, the sample was removed from the plate, the cap taken off and the slurry left to dry under ambient conditions before it was analysed by XRPD and DSC. This analysis showed compound 1 now to be in Form C, with a DSC melting point of 150° C. (onset).

### Example 3

### Inhibitory Action

In order to assess the inhibitory action of compound 1, the following assay was used to determine the IC₅₀ value.

Mammalian PARP, isolated from Hela cell nuclear extract, was incubated with Z-buffer (25 mM Hepes (Sigma); 12.5 mM MgCl₂ (Sigma); 50 mM KCl (Sigma); 1 mM DTT (Sigma); 10% Glycerol (Sigma) 0.001% NP-40 (Sigma); pH 7.4) in 96 well FlashPlates (TRADE MARK) (NEN, UK) and varying concentrations of said inhibitors added. The compound was diluted in DMSO and gave final assay concentrations of between 10 and 0.01 μM, with the DMSO being at a final concentration of 1% per well. The total assay volume per well was 40 μl.

After 10 minutes incubation at 30° C. the reactions were initiated by the addition of a 10 μl reaction mixture, containing NAD (5 μM), ³H-NAD and 30 mer double stranded DNA-oligos. Designated positive and negative reaction wells were done in combination with compound wells (unknowns) in order to calculate % enzyme activities. The plates were then shaken for 2 minutes and incubated at 30° C. for 45 minutes.

Following the incubation, the reactions were quenched by the addition of 50 μl 30% acetic acid to each well. The plates were then shaken for 1 hour at room temperature.

The plates were transferred to a TopCount NXT (TRADE MARK) (Packard, UK) for scintillation counting. Values recorded are counts per minute (cpm) following a 30 second counting of each well.

The % enzyme activity for the compound is then calculated using the following equation:

$$\% Inhibition = 100 - ( 100 \times \frac{( \begin{matrix}cpm of unknowns - \\ mean negative cpm\end{matrix} )}{( \begin{matrix}mean positive cpm - \\ mean neagative cpm\end{matrix} )} )$$

The IC₅₀ value (the concentration at which 50% of the enzyme activity is inhibited) were calculated, which are determined over a range of different concentrations, normally from 10 μM down to 0.001 μM.

Compound 1 has an IC₅₀ of about 5 nM.

### Potentiation Factor

The Potentiation Factor (PF₅₀) for compounds is calculated as a ratio of the IC₅₀ of control cell growth divided by the IC₅₀ of cell growth+PARP inhibitor. Growth inhibition curves for both control and compound treated cells are in the presence of the alkylating agent methyl methanesulfonate (MMS). The test compound was used at a fixed concentration of 30 nM and 200 nM. The concentrations of MMS were over a range from 0 to 10 μg/ml.

Cell growth was assessed using the sulforhodamine B (SRB) assay (Skehan, P., et al., (1990) New colorimetric cytotoxicity assay for anticancer-drug screening. J. Natl. Cancer Inst. 82, 1107-1112.). 2,000 HeLa cells were seeded into each well of a flat-bottomed 96-well microtiter plate in a volume of 100 μl and incubated for 6 hours at 37° C. Cells were either replaced with media alone or with media containing PARP inhibitor at a final concentration of 30 nM or 200 nM. Cells were allowed to grow for a further 1 hour before the addition of MMS at a range of concentrations (typically 0, 1, 2, 3, 5, 7 and 10 μg/ml) to either untreated cells or PARP inhibitor treated cells. Cells treated with PARP inhibitor alone were used to assess the growth inhibition by the PARP inhibitor.

Cells were left for a further 16 hours before replacing the media and allowing the cells to grow for a further 72 hours at 37° C. The media was then removed and the cells fixed with 100 μl of ice cold 10% (w/v) trichloroacetic acid. The plates were incubated at 4° C. for 20 minutes and then washed four times with water. Each well of cells was then stained with 100 μl of 0.4% (w/v) SRB in 1% acetic acid for 20 minutes before washing four times with 1% acetic acid. Plates were then dried for 2 hours at room temperature. The dye from the stained cells was solubilized by the addition of 10 μl of 10 mM Tris Base into each well. Plates were gently shaken and left at room temperature for 30 minutes before measuring the optical density at 564 nM on a Microquant microtiter plate reader.

Compound 1 as a PF₅₀ at 30 nM of 3.0, and at 200 nM of 15.

### Example 4

Cells from the Hela cell line known as KBA1, which expressed high P-glycoprotein (ABC1a and ABC1b transporter glycoproteins also known as MDR1a and MDR1b) and the matched non-P-glycoprotein expressing line known as KB31, were seeded onto 96 well tissue culture plates with 80 μl per well of 1.00×10⁴ cells/ml=800 cells/well [DMEM, 10% FBS, PSG] and left to adhere for 4 hours. After the incubation period 10 μl per well of 200 μM Verapamil (giving a final conc. of 20 μM) a known inhibitor of P-gp or vehicle media were added to various wells of the cell plates. The 96 well plates were left in the incubator for 1 hour prior to 10 μl of test compound (or the known substrate Etoposide as a reference control) or 10 μl PBS/1% DMSO vehicle (control wells) being added into either Verapamil containing or media control wells. The test compounds were tested over a range of different concentrations, normally from 100 μM down to 0.3 μM.

The cell plates were then incubated for 5 days prior to cell growth being assessed using the Sulforhodamine B (SRB) assay as described previously. The P-gp substrate activity for each compound was calculated using the cell growth activity of the tests compounds on the KBA1 cells in the presence or absence (control wells) of Verapamil. The Dose Modification Ratio (DMR) is calculated from the KBA1 where for each test compound a ratio of the IC₅₀ of the compound in the absence of Verapamil is divided by the IC₅₀ of cell growth in the presence of Verapamil. Compound that are not substrates for P-gp have a DMR of <1.5 while those compounds which are actively effluxed by P-gp generally show a DMR of >1.5 and more typically greater than 2.

Compound 1 has a DMR of 1.3.

1. 4-(4-Fluoro-3-(4-methoxypiperidine-1-carbonyl)benzyl)phthalazin-1(2H)-one as crystalline Form C.

2. The compound of claim 1 having at least one of the following characteristic peaks in a powder XRD using CuKa radiation: 19.3° and 18.5°.

3. The compound of claim 1 having the following characteristic peaks in a powder XRD using CuKa radiation: 19.3° and 18.5°.

4. The compound of claim 1 having at least four of the following characteristic peaks in a powder XRD using CuKa radiation: 19.3, 18.5, 18.9, 22.8, 10.5, 23.2°.

5. The compound of claim 1 having the following characteristic peaks in a powder XRD using CuKa radiation: 19.3, 18.5, 18.9, 22.8, 10.5, 23.2°.

6. The compound of claim 1, having an onset of melting at about 150.7° C.±1.0° C. when heated at 10° C. per minute in DSC.

7. A pharmaceutical composition comprising a compound according to claim 1 and a pharmaceutically acceptable carrier or diluent.

8. A compound according to claim 1 for use in a method of treatment of the human or animal body.

9. A compound according to claim 1 for the use in a method of inhibiting PARP in the treatment of the human or animal body.

10. The use of a compound according to claim 1 in the preparation of a medicament for inhibiting the activity of PARP.

11. The use of a compound according to claim 1 in the preparation of a medicament for the treatment of: vascular disease; septic shock; ischaemic injury; neurotoxicity; haemorraghic shock; viral infection; or diseases ameliorated by the inhibition of the activity of PARP.

12. The use of a compound according to claim 1 in the preparation of a medicament for use as an adjunct in cancer therapy or for potentiating tumour cells for treatment with ionizing radiation or chemotherapeutic agents.

13. Use of a compound according to claim 1 in the manufacture of a medicament for use in the treatment of cancer in an individual, wherein said cancer is deficient in HR dependent DNA DSB repair pathway.

14. Use according to claim 13, wherein said cancer comprises one or more cancer cells having a reduced or abrogated ability to repair DNA DSB by HR relative to normal cells.

15. Use according to claim 14, wherein said cancer cells have a BRCA1 or BRCA2 deficient phenotype.

16. Use according to claim 15, wherein said cancer cells are deficient in BRCA1 or BRCA2.

17. Use according to claim 13, wherein said individual is heterozygous for a mutation in a gene encoding a component of the HR dependent DNA DSB repair pathway.

18. Use according to claim 17, wherein said individual is heterozygous for a mutation in BRCA1 and/or BRCA2.

19. Use according to claim 13, wherein said cancer is breast, ovary, pancreas or prostate cancer.

20. Use according to claim 13, wherein said treatment further comprises administration of ionising radiation or a chemotherapeutic agent.
